(19) United States
(12) Patent Application Publication
(10) Pub. No.: US 2011/0282398 A1
(43) Pub. Date: Nov. 17, 2011

(54) Bone Screw Assembly and Instruments for Implantation of the Same

(76) Inventors: Tom OVERES, Langendorf (CH); Silas Zurschmiede, Langendorf (CH); Daniel Fluri, Langendorf (CH)

(21) Appl. No.: 13/103,697

(22) Filed: May 9, 2011

Related U.S. Application Data
(60) Provisional application No. 61/334,234, filed on May 13, 2010.

(51) Int. Cl. A61B 17/86

(52) U.S. Cl. 606/304; 606/305; 606/310

(57) ABSTRACT

A bone screw includes a screw shaft extending longitudinally along a screw axis and a screw head extending from a proximal end of the screw shaft and configured to be releasably coupled to a surgical tool. The bone screw also includes a through hole defining a through hole axis and extending through the screw head, the through hole axis intersecting the screw axis at an acute angle α, the through hole being adapted to receive therein a second screw and tapering from a first end at a proximal end of the screw head to a second end opening to an outer surface of the screw head.

### PRIORITY CLAIM

The present invention claims priority to U.S. Provisional Application Ser. No. 61/334,234 filed on May 13, 2010 and entitled, “Bone Screw Assembly and Implantation of the Same,” the entire disclosure of which is incorporated herein by reference.

### FIELD OF THE INVENTION

The present invention generally relates to bone screw assemblies and instruments for implantation of the same as well as to an associated method for implantation of the bone screw assembly using the instruments. More particularly, the invention relates to a bone screw including a second screw and insertion instruments for implantation of the same as well as to a method for implantation of the bone screw and the second screw by using the insertion instruments.

### BACKGROUND

In the field of orthopedic surgery bone fixation devices using bone screws are commonly used. These bone fixation devices include bone plates, intervertebral implants or intramedullary nails by means of which two or more bones or bone fragments are fixed relative to each other. Typically, the bone fixation devices comprise bone anchors mostly in the form of bone screws, pins or nails by means of which the bones or bone fragments are fixed to the bone plate, intervertebral implant or intramedullary nail and consequently fixed relative to each other. One problem that can arise in case of the above mentioned bone fixation devices is that the bone screws, pins or nails can for instance become dislodged in the bone or in the bone plate, intervertebral implant or intramedullary nail during normal movements of the patient.

Thus, there remains a need for an improved bone anchor device for use in bone fixation that allows to drill a hole for a securing screw into a bone under a particular angle with respect to the axis of a bone fastener and to insert the securing screw in a guided manner.

### SUMMARY OF THE INVENTION

The present invention relates to a bone screw with a screw head configured to be releasably coupled to a surgical instrument or tool such that the surgical instrument or tool is coaxially supported in a first position and can be pivoted in a guided manner about an axis extending diametrically to the screw head into a second position.

According to an exemplary embodiment of the present invention, the bone screw comprises a screw axis, a shaft to be anchored in a bone and screw head including a through hole with a through hole axis cutting the axis of the bone screw at an acute angle wherein the through hole is adapted to receive a second screw that can be anchored in the bone as well. The screw head comprises a transverse channel with a channel axis extending diametrically across the screw head and wherein the transverse channel is open at the rear end of the bone screw. The screw head of the bone screw further includes a recess which has—in at least a cross-section orthogonal to the channel axis—a circularly curved edge with a centre located at the point where the through hole axis cuts the screw axis.

One of the advantages of the bone screw according to the invention is that the configuration of the transverse channel and the recess permits an aiming guide with a complementarily configured tip to be attached to the screw head and rotated about a rotation axis from a first position aligned with the screw axis into a second position aligned with the through hole axis. Another advantage of the bone screw is the pivotable joining of the screw head and the aiming guide. The aiming guide can be inserted into the incision coaxially to the screw axis of the bone screw by using a tissue protection tube and/or a guide wire and attached to the screw head. After removing the tissue protection tube and/or the guide wire the aiming guide can be rotated about the rotation axis. In addition, the aiming guide can then be rotated into a second position in which the longitudinal axis of the aiming guide is aligned with the through hole axis of the through hole in the screw head so that a bore hole for the second screw can be drilled into the bone which is exactly aligned with the through hole in the screw head; the bore hole for the second screw can be drilled in a completely guided manner and the second screw can be positioned in a guided manner by means of the aiming guide. Another advantage is that the transverse channel and the recess allow to attach a complementarily formed tip of a screwdriver to the bone screw in such a manner that the screwdriver is positioned coaxially to the screw axis of the bone screw.

In an exemplary embodiment of the bone screw, the through hole axis cuts the screw axis at a depth T>0 measured from the rear end of the bone screw towards the screw shaft.

In another exemplary embodiment of the bone screw, the recess has a constriction at the rear end of the bone screw. Thus, the recess forms a female connector for a snap-lock connection with a respective male connector arranged at a surgical instrument or tool.

In a further exemplary embodiment of the bone screw, the through hole comprises an internal thread, preferably a conical internal thread. This allows the advantage that the second screw can be firmly connected to the screw head of the bone screw.

In another exemplary embodiment of the bone screw, the internal thread has a thread pitch P and a threaded length L_{T} and wherein the ratio L_{T}/P is minimum 2.0, preferably minimum 2.3. This configuration of the internal thread allows a rigid and angularly stable anchorage of the screw head of the second screw in the screw head of the bone screw.

In yet another exemplary embodiment of the bone screw, the recess has a spherical shape with a radius of the sphere R. This configuration of the recess allows a surgical instrument or tool to be pivoted about an axis which extends through the point where the through hole axis and the screw axis intersect so that the instrument or tool can be pivoted from a first position aligned with the screw axis to a second position aligned with the through hole axis.

In a further exemplary embodiment, the bone screw further comprises a second screw insertable into the through hole coaxially to the through hole axis.

In another exemplary embodiment of the bone screw, the second screw has a conically threaded head engagable with the conical internal thread in the through hole.

In yet a further exemplary embodiment of the bone screw, the recess includes a depression traversing the constriction and forming a wall portion with the shape of a surface section of a cylinder, cone or prism the axis of which coincides with the through hole axis. By means of the depression a stop for the rotation of an instrument or tool inserted in the recess in the screw head of the bone screw is provided so that the instrument or tool can be exactly aligned with the through hole for the second screw.

In another exemplary embodiment of the bone screw, the angle α amounts to minimum 10°, preferably to minimum 20°.

In yet another exemplary embodiment of the bone screw, the angle α amounts to maximum 70°, preferably to maximum 35°.

In again another exemplary embodiment of the bone screw, the transverse channel has a U-shape in a cross-section orthogonal to the channel axis. The U-shaped channel can have a semicircular bottom with a radius of curvature r_{C}, wherein the centre of the semicircular edge of the transverse channel is located on the channel axis. The channel axis can be located at a depth T_{C} measured from the rear end of the bone screw towards the screw shaft, wherein the depth T_{C} is equal or greater than the depth T of the point where the through hole axis cuts the screw axis. In a particular configuration of the transverse channel the channel axis cuts the screw axis through the point where the through hole axis and the screw axis intersect, i.e. T_{C}=T. In this case the semicircular bottom defines a seat coaxially to the recess for rotatably receiving cylindrical pins of an aiming guide which have a pin diameter equal to twice the radius of curvature r_{C} of the semicircular bottom of the transverse channel. In case of a spherical recess the rotatable movement of the aiming guide is limited to a uniaxial pivot movement due to the pins engaging the transverse channel.

In a further exemplary embodiment of the bone screw, the channel axis cuts the screw axis through the point at which the screw axis and the through hole axis intersect.

In yet another exemplary embodiment of the bone screw, the screw head of the bone screw comprises an external thread designed in such a manner that the bone screw can be counter-sunk in a bone. The external thread on the screw head is preferably_conical so that it allows to countersink the screw head in the bone. This configuration is particularly useful if the bone screw is used as a locking screw for an intramedullary nail.

In another exemplary embodiment of the bone screw, the screw head of the bone screw has a longitudinal slot so that the screw head is radially elastically expandable. The screw head can have the shape of a segment of a sphere so that the bone screw can be inserted into a complementarily shaped hole in a bone plate or other implant under a surgeon desired angle. Once the bone screw is correctly positioned the second screw can be inserted until the head of the second screw expands the screw head of the bone screw in the hole so allowing to secure the bone screw in a surgeon selected angle relative to a bone plate or other implant.

In accordance with another aspect of the present invention, a screwdriver is provided for screwing the above bone screw into a bone. The screwdriver essentially comprises a male connector terminally arranged at the front end which is suitable to be coupled to the recess in the screw head of the bone screw. Further, the connector includes a tip constricting towards the front end of the screwdriver and two driving protrusions diametrically projecting over the tip in either direction and defining a central axis which extends orthogonal to the longitudinal axis of the screwdriver. The driving protrusions fit in the transverse channel in the screw head of the bone screw. In at least a cross-section orthogonal to the central axis the tip has a circularly curved periphery with a radius R and a centre located on the longitudinal axis. The driving protrusions can have the shape of pins or blades. In case of blade-shaped driving protrusions the tips of the blades define the central axis. In case of pin-shaped driving protrusions the axes of the pins define the central axis.

In an exemplary embodiment, the screwdriver further comprises a longitudinal slot extending parallel to the longitudinal axis and which is open at the front end so that the connector is radially elastically compressible. Further, the tip has a constriction towards the shaft which forms at least in a cross-section orthogonal to the central axis a curved contact shoulder. Thus, the connector forms a male connector for a snap-lock connection with a respective female connector arranged at the bone screw.

In a further exemplary embodiment of the screwdriver, the tip has a spherical shape with a radius of the sphere R and with a centre located on the longitudinal axis.

In a further exemplary embodiment of the screwdriver, the two driving protrusions are circular-cylindrically shaped wherein the central axis orthogonally cuts the longitudinal axis through the centre of the spherical tip.

In another exemplary embodiment of the screwdriver, the male connector further comprises an axial stop located at a distance T measured from the central axis towards the shaft so that the stop contacts the rear end of the bone screw when the connector is coupled to the recess in the screw head. Thus, the screwdriver is kept exactly coaxially to the screw axis of the bone screw when the stop abuts the rear end of the bone screw.

In yet another exemplary embodiment of the screwdriver, the male connector further includes a nose projecting over the tip in a direction towards the front end and at an acute angle with respect to the longitudinal axis of the screwdriver. This configuration allows the advantage that the screwdriver can only be inserted in one orientation into the seat in the screw head of the bone screw.

In again another exemplary embodiment, the screwdriver further comprises a coaxial through bore penetrating through the shaft and the male connector and having an internal thread for engaging an external thread arranged on a locking pin which is insertable in the through bore in such a manner that the locking pin can be advanced towards the front end of the screwdriver to prevent the tip from radially collapsing.

In accordance with a further aspect of the present invention, an aiming guide is provided for drilling a hole in the bone the axis of which coincides with the through hole axis of the through hole in the screw head of the bone screw. The aiming guide essentially comprises a guide sleeve, a coaxial through bore and a male connector terminally arranged at the front end which is suitable to be coupled to the recess in the screw head of the bone screw. The connector includes a tip a width of which decreases towards the front end of the aiming guide and two pins diametrically projecting over the tip in either direction and coaxially arranged on a central axis which extends orthogonal to the longitudinal axis. In at least a cross-section orthogonal to the central axis the tip has a circularly curved periphery with a radius R and a centre located on the longitudinal axis.

In an exemplary embodiment, the aiming guide further comprises a longitudinal slot extending parallel to the longitudinal axis and which is open at the front end so that the connector is radially elastically compressible. Additionally, a curved contact shoulder is formed at the proximal end of the tip. Specifically, the curved contact shoulder is formed by a constriction at the proximal end of the tip (adjacent the guide sleeve) where a cross-sectional area of the tip in a plane orthogonal to the central axis of the tip is reduced relative to a maximum diameter portion of the tip distal thereto. This configuration allows the connector to operate as a male connector for a snap-lock connection with a respective female connector arranged in the bone screw.

In another exemplary embodiment of the aiming guide, the tip has a spherical shape with a radius of the sphere R and with a center located on the longitudinal axis.

In a further exemplary embodiment of the aiming guide, the two pins are circular-cylindrically shaped and wherein the central axis orthogonally cuts the longitudinal axis through the centre of the spherical tip. The circular cylindrical pivot pins are coaxially and rotatably insertable in the transverse channel.

In yet another exemplary embodiment of the aiming guide, a cylindrical or conical collar is arranged between the tip and the guide sleeve coaxially to the longitudinal axis of the aiming guide.

In again another exemplary embodiment of the aiming guide, the collar has a radius r≦R.

In a further exemplary embodiment, the aiming guide further comprises a drill guide which can be inserted into the through hole.

In another exemplary embodiment of the aiming guide, the drill guide has a conical tip. The conical tip is shaped in such a manner that it fits into the tapered through hole in the bone screw. This allow to lock the drill guide aligned to the through hole axis of the through hole for the second screw.

In accordance with yet another aspect of the present invention, an assembly including a bone screw, a screwdriver and an aiming guide is provided. This assembly can be used if a standard locking screw which is commonly available can be inserted into the through hole in the bone screw.

In accordance with again another aspect of the present invention, an assembly including a bone screw, a tissue protection tube, an aiming guide and a screwdriver is provided.

In accordance with still another aspect of the present invention, a method for bone fixation using an intramedullary nail including a number of proximal and distal locking holes and a number of bone screws with a second screw each is provided. The method essentially comprises the steps of:
a) performing an incision into the tissue surrounding a bone to be treated;
b) positioning an intramedullary nail in the bone;
c) coupling an aiming device to the intramedullary nail;
d) inserting a tissue protection tube into a selected guide bore in the aiming device coaxially to one of the locking holes;
e) drilling a first bore hole into a bone for insertion a bone screw by using the aiming device, wherein the first bore hole is aligned with the selected proximal or distal locking hole;
f) coupling a bone screw to the connector of the screwdriver;
g) advancing the bone screw through the tissue protection tube;
h) screwing the bone screw into the bone using the screwdriver;
i) removing the screwdriver;
j) inserting the aiming guide through the tissue protection tube;
k) attaching the aiming guide to the bone screw in such a manner that the longitudinal axis of the aiming guide is aligned with the screw axis of the bone screw;
l) removing the tissue protection tube;
m) repeating steps d) to l) until a bone screw each is inserted in all or in the selected proximal and/or distal locking holes of the intramedullary nail;
n) removing the aiming device from the intramedullary nail;
o) pivoting the aiming guide about the central axis until the collar of the aiming guide abuts a stop in the recess in the screw head of the bone screw so that the longitudinal axis of the aiming guide is aligned with the through hole axis of the through hole in the bone screw;
p) inserting the drill guide into the through bore in the aiming guide;
q) drilling a second bore hole into the bone using the drill guide as a guide for the drill bit;
r) removing the drill guide;
s) inserting the second screw through the through bore in the aiming guide;
t) advancing the second screw into the bone;
u) removing the aiming guide;
v) repeating steps o) to u) until a second screw each is anchored in the bone passing through the through hole of each of the bone screws; and
w) closing the incision.

Instead of subsequently performing steps d) to l) for one bone screw and repeating the sequence for each bone screw to be inserted each step can be repeated for all or for the selected number of bone screws to be inserted. Similarly, each step for inserting the second screw can be repeated for all or for the selected number of bone screws instead of subsequently performing steps o) to u) for one bone screw and repeating the sequence for each bone screw to be inserted.

In an exemplary embodiment, the stop is formed by the wall portion of the depression in the recess in the screw head of the bone screw.

### BRIEF DESCRIPTION OF THE DRAWINGS

An exemplary embodiment of the present invention will be described in the following by way of example and with reference to the accompanying drawings in which:

FIG. 1 illustrates a longitudinal section of an embodiment of the bone screw according to the invention;

FIG. 2 illustrates a lateral view of the embodiment of the bone screw of FIG. 1;

FIG. 3 illustrates a perspective view of an embodiment of the screwdriver according to the invention;

FIG. 4 illustrates a longitudinal section of the embodiment of the screwdriver of FIG. 3;

FIG. 5 illustrates a lateral view of the embodiment of the screwdriver of FIG. 3;

FIG. 6 illustrates a lateral view of the embodiment of the screwdriver of FIG. 3 which is orthogonal to the lateral view of FIG. 5;

FIG. 7 illustrates a lateral view of another embodiment of the screwdriver according to the invention;

FIG. 8 illustrates a lateral view of the embodiment of the screwdriver of FIG. 7 which is orthogonal to the lateral view of FIG. 7;

FIG. 9 illustrates a partial section through the embodiment of the screwdriver of FIGS. 7 and 8 and a bone screw attached thereto;

FIG. 10 illustrates a perspective view of an embodiment of the aiming guide according to the invention;

FIG. 11 illustrates a longitudinal section through the embodiment of the aiming guide of FIG. 10 and a bone screw coaxially attached thereto;

FIG. 12 illustrates a longitudinal section through the embodiment of the aiming guide of FIG. 10 and a bone screw attached thereto under the angle α;

FIG. 13 illustrates a longitudinal section through the embodiment of the aiming guide of FIG. 10 and a bone screw attached thereto under the angle α and together with a drill guide inserted in the aiming guide and a drill bit;

FIG. 14 illustrates a section through the aiming device and the drill guide of FIG. 13;

FIG. 15 illustrates a longitudinal section through the embodiment of the aiming guide of FIG. 10 and a bone screw attached thereto under the angle α and together with a second screw inserted in the aiming guide;

FIG. 16 illustrates a longitudinal section through the embodiment of the aiming guide of FIG. 10 and a bone screw attached thereto under the angle α and together with a second screw firmly secured in the through hole in the screw head of the bone screw; and

FIG. 17 illustrates an intramedullary nail together with a bone screw and a second screw according to an embodiment of the method for bone fixation according the invention.

FIG. 18 illustrates a lateral view of a system according to an alternate embodiment of the present invention, in a first configuration.

FIG. 19 illustrates a lateral view of the system of FIG. 18, in a second configuration.

FIG. 20 illustrates a cross-sectional lateral view of the system of FIG. 18, in the second configuration.

FIG. 21 illustrates an enlarged cross-sectional lateral view of a portion of the system of FIG. 18.

### DETAILED DESCRIPTION

The present invention may be further understood with reference to the following description and the appended drawings, wherein like elements are referred to with the same reference numerals. The present invention relates to bone screw assemblies and instruments for implantation of the same as well as to an associated method for implantation of the bone screw assembly using the instruments. In particular, the invention relates to a system and method facilitating implantation of a first bone screw, including a through hole extending through a head portion thereof along a through hole axis, and a second screw inserted into the through hole along the through hole axis such that the first and second bone screws are implanted into a bone in a stable configuration.

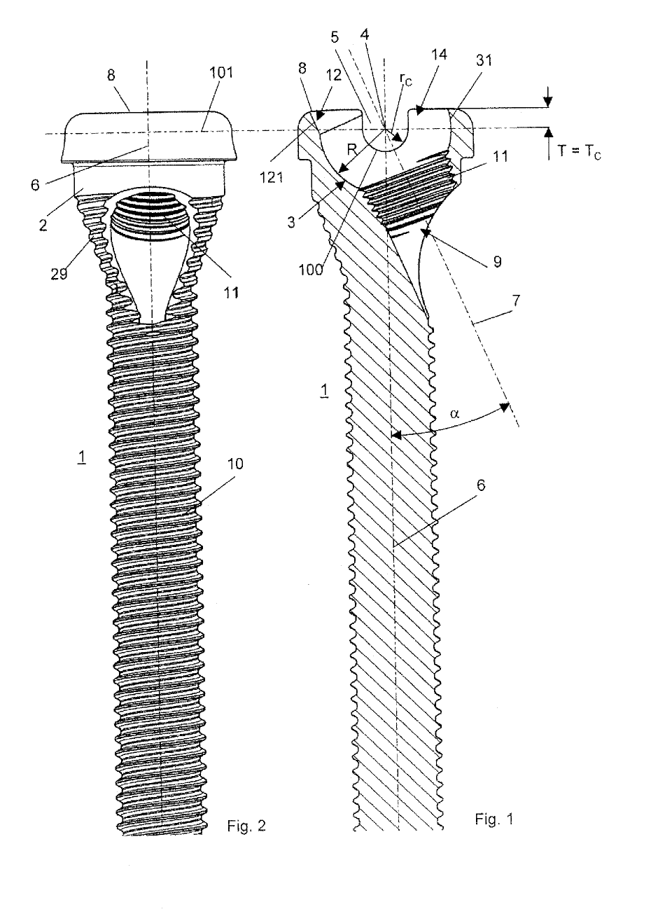

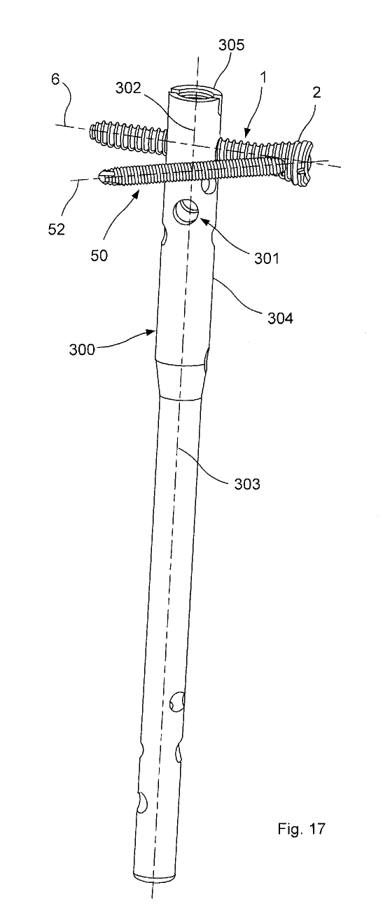

FIGS. 1 and 2 illustrate an embodiment of the bone screw 1 with a screw head 2 comprising a conical external thread 29. The bone screw 1 includes a screw axis 6, a threaded shaft 10, a screw head 2 and a rear end 8 at a proximal end thereof. The screw head 2 comprises a through hole 9 penetrating through the screw head 2 and having a through hole axis 7 cutting the screw axis 6 under an acute angle α. The through hole axis 7 cuts the screw axis 2 at a depth T measured from the rear end 8 of the bone screw 1. The through hole 9 has a conical internal thread 11. A second screw 50 (FIGS. 16 and 17) can be inserted into the through hole 9 coaxially to the through hole axis 7. The second screw 50 has a conically threaded head 51 which is engagable with the conical internal thread 11 in the through hole 9. The screw head 2 includes a concave seat 14 for releasably coupling a surgical instrument or tool to the bone screw 1. The concave seat 14 comprises a transverse channel 5 and a centrally located recess 3. The transverse channel 5 comprises a channel axis 101 located at a depth T_{C} measured from the rear end 8 of the bone screw 1 and diametrically extending across the screw head 2. The channel axis 101 cuts the screw axis 6 through the point where the through hole axis 7 cuts the screw axis 6. Further, the transverse channel 5 is open at the rear end 8 of the bone screw 1 and the transverse channel 5 has a U-shaped cross-section with a semicircular bottom 100 orthogonal to the channel axis 101. The semicircular bottom 100 has a radius of curvature r_{C} wherein a center of an edge the semicircular bottom 100 of the transverse channel 5 is located on the channel axis 101. The depth T_{C} is equal to the depth T of the point where the through hole axis 7 cuts the screw axis 1. The semicircular bottom 100 defines a seat coaxially to the recess 3 for rotatably receiving cylindrical pins 25 of an aiming guide 23 (FIG. 10) which have a pin diameter equal to twice the radius of curvature r_{c} of the semicircular bottom 100 of the transverse channel 5.

The recess 3 has a spherical shape with a radius of the sphere R and a centre 4 coinciding with the point at which the screw axis 6 and the through hole axis 7 intersect. So the recess 3 forms a pivot bearing for rotatably supporting and guiding a complementarily spherically shaped male connector 232 of an aiming guide 23 (FIG. 10). Due to the facts that the channel axis 101 cuts the screw axis 6 at the point where the centre 4 of the spherically shaped recess 3 is located on the screw axis 6 and that the pivot pins 25 of the aiming guide 23 fit in the transverse channel 5 rotatably about the channel axis 101 the polyaxial pivot bearing formed by the ball-and-socket joint is limited to an uniaxial pivot bearing. When the aiming guide 23 is coupled to the bone screw 1 the aiming guide 23 can only pivot about the channel axis 101 which is orthogonal to a plane defined by the screw axis 6 and the through hole axis 7 of the through hole 9 for the second screw 50. This allows to position the aiming guide 23 in a first position coaxial to the screw axis 6 of the bone screw 1 and in a second position coaxial to the through hole axis 7 of the through hole 9 for the second screw 50. Thus, it will be understood by those of skill in the art, that the second screw 50 may be precisely inserted into the through hole 9 along through hole axis 7, increasing a stability of the screws 1, 50 in situ.

Furthermore, the recess 3 has a constriction 31 at the rear end 8 of the bone screw 1 so that the recess 3 forms a female connector for a snap-lock connection. Additionally, the recess 3 includes a depression 12 which forms a wall portion 121 with the shape of a surface section of a circular cylinder with a radius r≦R. The axis of the circular cylinder coincides with the through hole axis 7 of the through hole 9. The depression 12 forms a stop for the rotation of an aiming guide 23 about the channel axis 101 when the aiming guide 23 is coupled to the screw head 2 of the bone screw 1. By means of the stop the aiming guide 23 can be exactly aligned with the through hole 9.

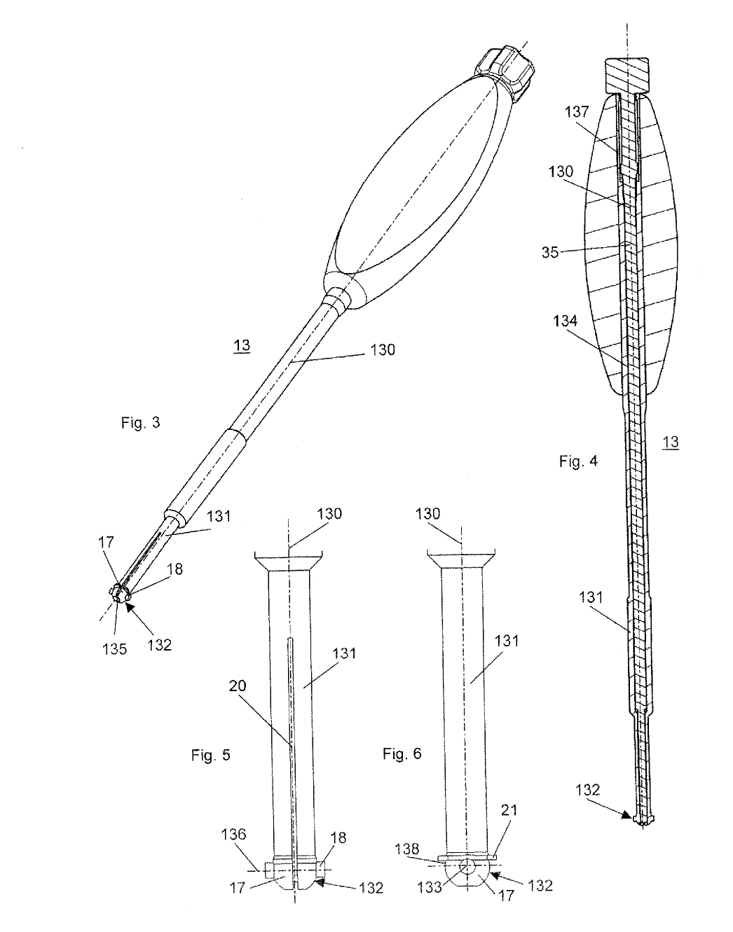

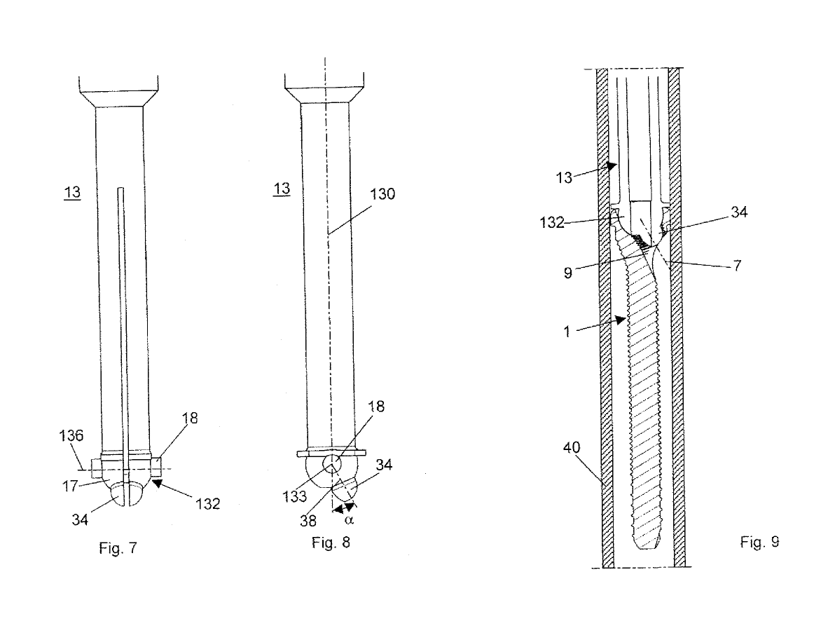

FIGS. 3 to 6 illustrate an embodiment of the screwdriver 13 to be used with the bone screw 1 according to FIGS. 1 and 2. The screwdriver 13 comprises a longitudinal axis 130, a shaft 131, a front end 135 at a distal end thereof and a male connector 132 which can be coupled to the above embodiment of the bone screw 1. In order to releasably couple the screwdriver 13 to the bone screw 1 the connector 132 is essentially complementarily formed to the concave seat 14 in the screw head 2 of the bone screw 1. The connector 132 includes a partially spherical tip 17 the cross-sectional area of which in a plane perpendicular to the longitudinal axis decreases toward the shaft 131 (i.e., toward the front end 135). Thus, a cross-sectional area of the spherical tip 17 at an end adjacent to the shaft 131 and at the front end 135 is smaller than a cross-sectional area of a mid-section of the spherical tip 17. The tapering of the spherical tip 17 towards the shaft 131 forms a curved contact shoulder 138 which abuts the constriction 31 of the recess 3 at the rear end 8 of the bone screw 1. The screwdriver 13 further comprises a longitudinal slot 20 open at the front end 135 to form the tip 17 as an elastic male connector for a snap-lock connection between the tip 17 and the recess 3 in the screw head 2 of the bone screw 1. The longitudinal slot 20 is arranged orthogonal to a plane defined by the longitudinal axis 130 and the central axis 136 and penetrates through the shaft 131. The connector 132 further includes two driving protrusions 18 extending laterally from the spherical tip 17 in either direction and which are coaxially arranged on a central axis 136. The driving protrusions 18 are circular-cylindrically shaped with a cylinder axis coinciding with the central axis 136. The connector 132 additionally comprises an axial stop 21 which is located between the shaft 131 and the connector 132 at a distance T measured from the central axis 136 towards the shaft 131. The axial stop 21 abuts the rear end 8 of the bone screw 1 allowing to keep the screwdriver 13 exactly coaxially to the screw axis 6 of the bone screw 1. The shaft 131 and the connector 132 comprise a coaxial through bore 134 with an internal thread 137 for engaging an external thread arranged on a locking pin 35 (FIG. 4) which is insertable in the through bore 134 in such a manner that the locking pin 35 can be advanced towards the front end 135 of the screwdriver 13 in order to prevent the tip 17 from radially collapsing so that it can be firmly kept in the recess 3 in the screw head 2 of the bone screw 1.

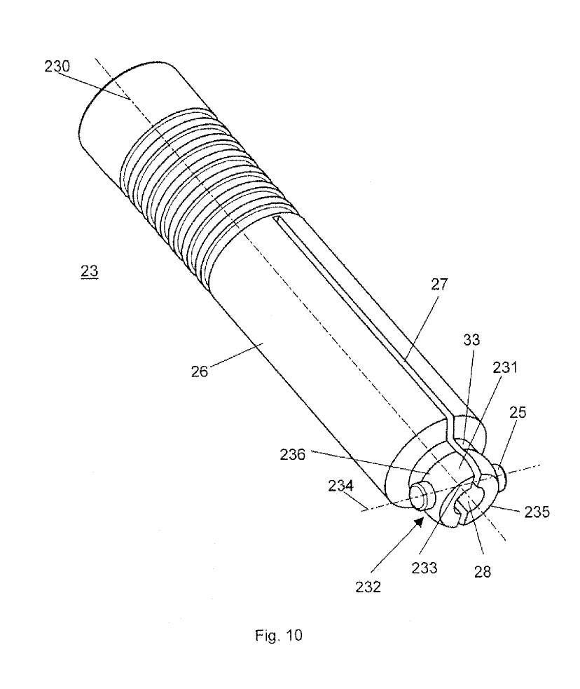

The embodiment of the screwdriver 13 illustrated in FIGS. 7 to 9 differs from the embodiment of FIGS. 3 to 6 only therein that the male connector 132 further includes a nose 34 extending from the tip 17 in a direction towards the front end 135. The nose 34 has a nose axis 38 extending under the angle α with respect to the longitudinal axis 130 of the screwdriver 13. Thus, the screwdriver 13 can be coupled to the bone screw 1 in only one rotative position, namely the one position where the nose 34 engages the through hole 9 in the bone screw 1 in such a manner that the nose axis 38 coincides with the through hole axis 7.

FIG. 10 illustrates an embodiment of the aiming guide 23 to be used with the bone screw 1 according to FIGS. 1 and 2. The aiming guide 23 comprises a longitudinal axis 230, a coaxial through bore 28, a guide sleeve 26, a front end 235 and a male connector 232 terminally arranged at the front end 235. To releasably couple the aiming guide 23 to the bone screw 1 the connector 232 is essentially complementarily formed to the seat 14 in the screw head 2 of the bone screw 1. The connector 232 includes a spherically shaped tip 231 which tapers inward toward the front end 235 and toward the guide sleeve 26 forming a ball-and-socket joint with the recess 3 of the above described embodiment of the bone screw 1. The spherically shaped tip 231 has a radius of the sphere R and a centre 233 located on the longitudinal axis 230. Additionally, the connector 232 includes two pins 25 diametrically projecting over the tip 231 in either direction and which are coaxially arranged on a central axis 234 which extends orthogonal to the longitudinal axis 230 and which cuts the longitudinal axis 230 through the centre 233 of the spherically shaped tip 231. The pins 25 are circular-cylindrically shaped with a cylinder axis coinciding with the central axis 234 so as to form axles coaxially and rotatably insertable in the transverse channel 5 in the screw head 2 of the bone screw 1. The aiming guide 23 further comprises a longitudinal slot 27 open at the front end 235 to form the tip 231 as an elastic male connector for a snap-lock connection between the aiming guide 23 and a the spherical recess 3 in the screw head 2 of the bone screw 1. The longitudinal slot 27 is arranged orthogonal to a plane defined by the longitudinal axis 230 and the central axis 234 and penetrates through the guide sleeve 26. Between the tip 231 and the guide sleeve 26 a cylindrical collar 33 is arranged coaxially to the longitudinal axis 230 and which has a radius r≦R.

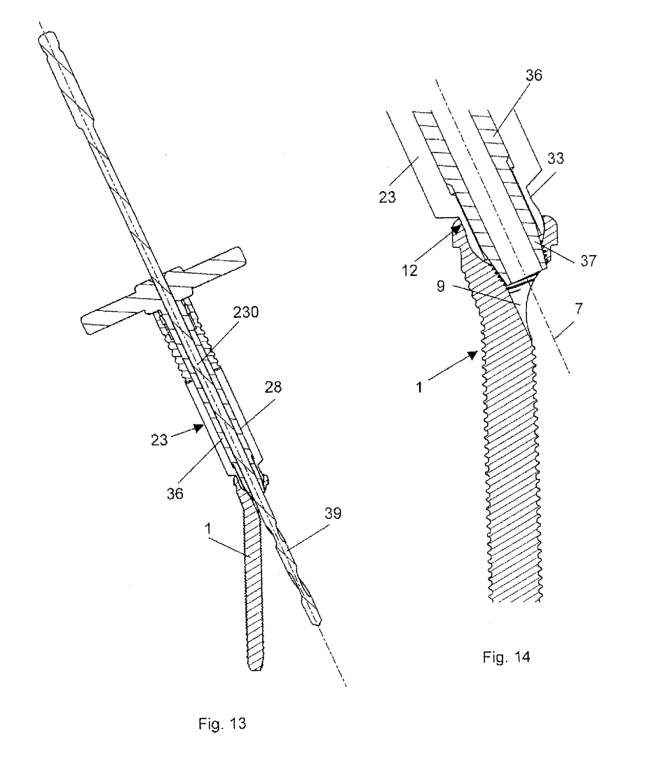

As illustrated in FIGS. 13 and 14 a drill guide 36 can be inserted in the through bore 28 in the aiming guide 23. The drill guide 36 has a conical tip 37 which fits into the tapered through hole 9 in such a manner that the drill guide 36 is exactly aligned with the through hole axis 7 of the through hole 9 in the bone screw 1.

FIGS. 11 to 17 show an embodiment of the method for bone fixation by using an intramedullary nail 300 and bone screws 1 along with second screws 50, which are briefly described in the following section. The intramedullary nail 300 comprises a nail axis 303, a proximal end 305, a peripheral surface 304, a number of proximal locking holes 301 with a most proximal locking hole 302 and a number of distal locking holes 306. The proximal and distal locking holes 301, 306 extend transverse to the nail axis 303. The intramedullary nail 300 is inserted into the intramedullary canal of a long bone in such a manner that the portion of the intramedullary nail 300 containing the distal locking holes 306 is located in a distal bone fragment and the portion containing the proximal locking holes 301 is located in the proximal bone fragment. In order to lock the intramedullary nail 300 in the bone a bone screw 1 each is driven through all or a number of selected proximal and distal locking holes 301, 306. Using the bone screw 1 according to the invention the bone screws 1 can be driven through all or the selected proximal and distal locking holes 301, 306 and the second screws 50 can be anchored in the bone. It should be appreciated that instead of driving the bone screws 1 into the proximal and distal locking holes 301, 306 the second screws 50 could be driven into the proximal and distal locking holes 301, 306 and the bone screws 1 could be anchored in the bone.

The method for inserting the bone screws 1 into the nail 300 and anchoring the second screws 50 into the bone comprises the steps of making an incision into the tissue surrounding a bone to be treated and positioning an intramedullary nail 300 in the bone. An aiming device (not shown) to the proximal end 305 of the intramedullary nail 300, wherein the aiming device has guide bores for inserting guide sleeves and/or tissue protection tubes 40 coaxially to each of all or of a number of selected proximal and/or distal locking holes 301, 306. A tissue protection tube 40 (FIGS. 9 and 11) is inserted into a selected guide bore in the aiming device coaxially to one of the proximal and distal locking holes 301, 306 until the front end of the tissue protection tube 40 contacts the surface of the bone. A first bore hole is then drilled into a bone for insertion of a bone screw 1 through the selected proximal or distal locking hole 301, 306 by using the aiming device, wherein the first bore hole is aligned with the selected proximal or distal locking hole 301, 306. Further, the bore hole extends on either side of the intramedullary nail 300 in such a manner that a bone screw 1 can penetrate through the selected proximal or distal locking hole 301, 306 of the intramedullary nail 300 when the bone screw 1 is anchored in the bone. The bone screw 1 is coupled to the connector 132 of the screwdriver 13 by using the snap-lock connection between the bone screw 1 and the screwdriver 13 and advancing the bone screw 1 through the tissue protection tube 40. The bone screw 1 is then screwed into the bone using the screwdriver 13. Once the bone screw 1 is screwed into the bone, the screwdriver 13 may be removed. The above-described steps may be repeated until a bone screw 1 has been inserted into all of the desired proximal and/or distal locking holes 301, 302.

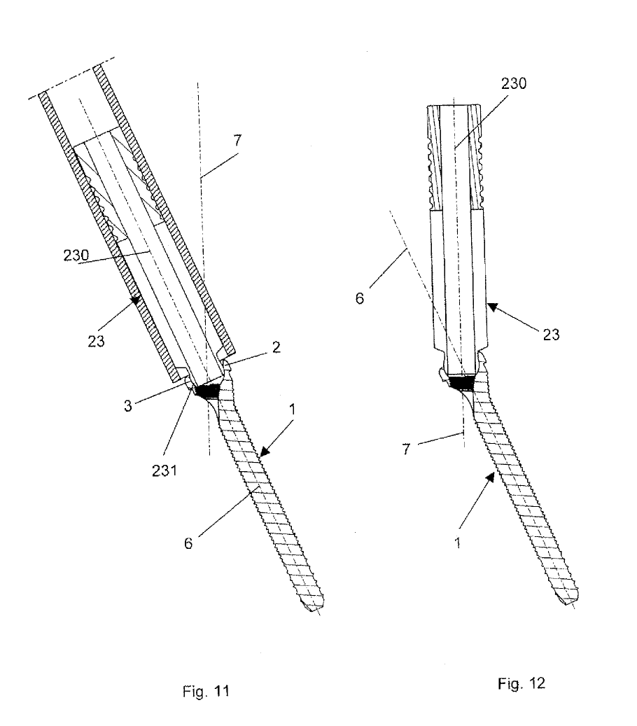

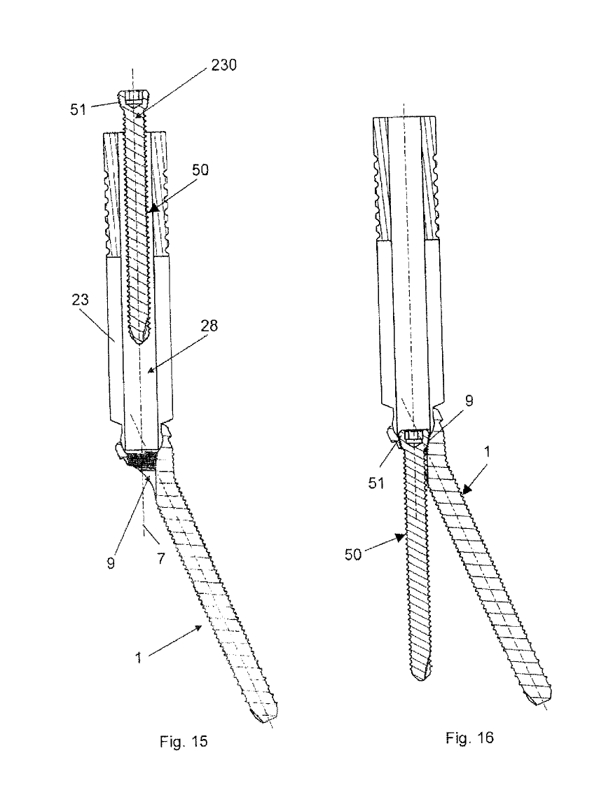

The aiming guide 23 is then inserted through the protection tube 40, as shown in FIG. 11, to attach the aiming guide 23 to the bone screw 1 using the snap-lock connection between the tip 231 of the aiming device 23 and the recess 3 in the screw head 2 of the bone screw 1 such that the longitudinal axis 230 of the aiming guide 23 is aligned with the screw axis 6 of the bone screw 1. The tissue protection tube 40 and the aiming device may be removed and the and the aiming guide 23 pivoted about the central axis 234 defined by the pins 25 arranged at the connector 232 of the aiming guide 23 until the collar 33 of the aiming guide 23 abuts a stop in the recess 3 in the screw head 2 of the bone screw 1 so that the longitudinal axis 230 of the aiming guide 23 is aligned with the through hole axis 7 of the through hole 9 in the bone screw 1 (FIG. 12). The stop is formed by the wall portion 121 of the depression 12 in the recess 3 in the screw head 2 of the bone screw 1. The drill guide 36 is then inserted into the through bore 28 in the aiming guide 23 (FIG. 13) and a second bore hole is drilled into the bone using the drill guide 36 as a guide for the drill bit 39. Once the second bore hole has been drilled, the drill guide 36 is removed and the second screw 50 is inserted through the through bore 28 in the aiming guide 23 (FIG. 15) and advanced through the second screw 50 into the bone (FIG. 16). The aiming guide 23 may then be removed and the steps described above repeated until a second screw 50 each is anchored in the bone passing through the through hole 9 of each of the bone screws 1. Once all of the desired bone screws 1 and second screws 50 have been inserted into the bone, the incision may be closed.

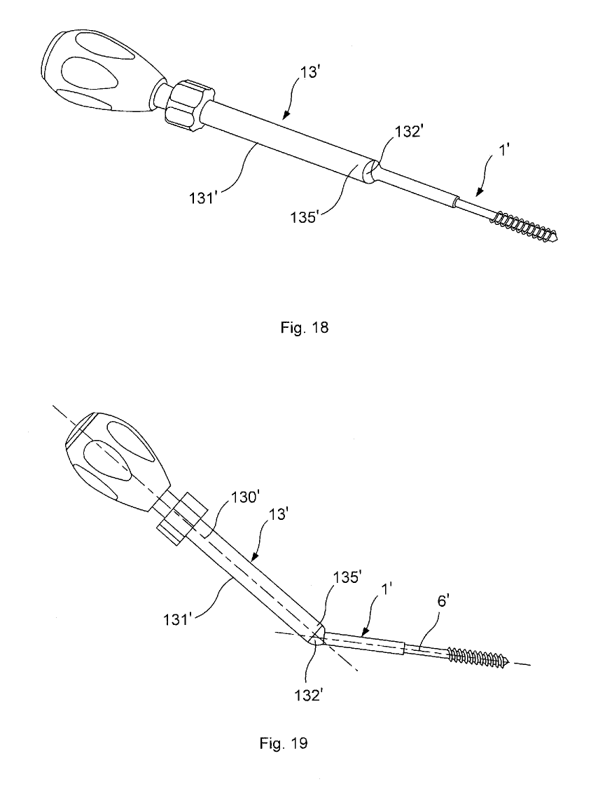

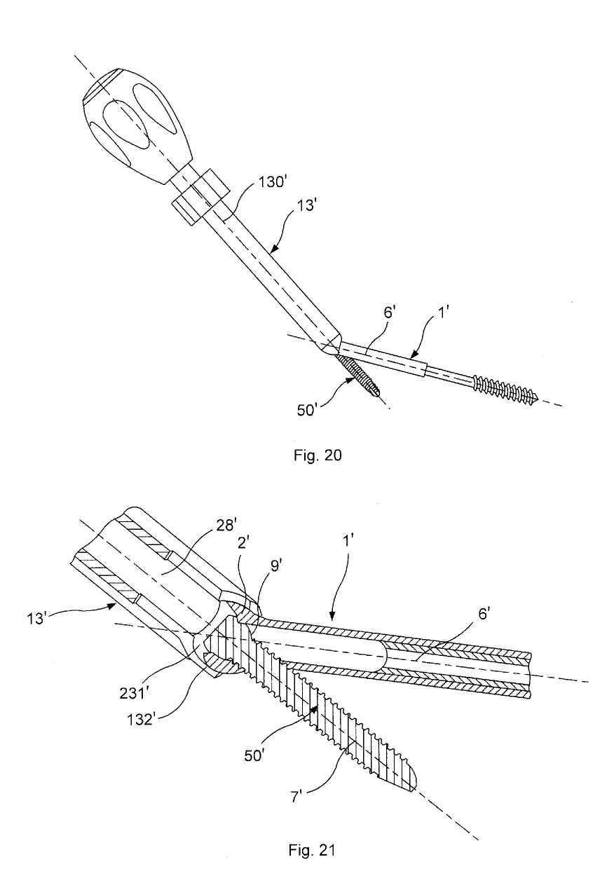

As shown in FIGS. 18-21, an alternate embodiment of the assembly of the present invention is substantially similar to the assembly described above in regard to FIGS. 1-17, comprising a first bone screw 1′, a second bone screw 50′ and a screwdriver 13′. The screwdriver 13′, however, combines elements of the screwdriver 13 and the aiming guide 23, as described above, such that two separate devices are not required for insertion of the first bone screw 1′ and for aiming the second bone screw 50′. In addition, the screwdriver 13′ includes a connector 132′ at a distal end 135′ thereof, which extends around a head 2′ of the first bone screw 1′ rather than within a recess thereof.

The first bone screw 1′ extends along a first axis 6′ and includes a head 2′, which has an exterior surface that is at least partially spherical. The exterior surface may also include portions that are substantially planar permitting a torsional force to be applied thereto via the screwdriver 13′. Similarly to the bone screw 1, the first bone screw 1′ includes a through hole 9′ extending along a second axis 7′ to receive the second screw 50′ therein. The second screw 50′ is substantially similar to the second screw 50′ described above.

The screwdriver 13′ includes a shaft 131′ extending along a longitudinal axis 130′ with a connector 132′ formed at the distal end 135′ thereof. The screwdriver 13′ also includes a channel 28′ extending therethrough along the longitudinal axis 130′ sized and shaped to permit the second bone screw 50′ to be inserted therethrough. The connector 132′ includes a partially spherical interior surface 231′ sized and shaped to receive the head 2′ of the first bone screw 1′ therein. In one embodiment, the connector 132′ may be keyed (e.g., include planar portions corresponding to the planar portions of the head 2′) permitting the screwdriver 13′ to apply torsional forces to the bone screw 1′ while also permitting the first bone screw 1′ to pivot with respect to the screwdriver 13′ via the partially spherical surfaces of the connector 132′ and the head 2′. The interior surface 231′ may receive the head 2′ via, for example, a snap fit.

In an alternative embodiment, the head 2′ may include pins extending radially outward therefrom, which are substantially similar to the pins 25 of the connector 232 of the aiming guide 23, and the connector 132′ may include a transverse channel diametrically extending thereacross similarly to the channel 5 of the bone screw 1, as described above. It will be understood by those of skill in the art that such a configuration also permits the first bone screw 1′ to be rotated via the screwdriver 13′ while also permitting the first bone screw 1′ to be pivoted relative thereto.

The first and second bone screws 1′, 50′ and the screwdriver 13′ may be used in a manner substantially similar to the method described above. In particular, the first bone screw 1′ may be inserted into a desired one of the proximal and/or distal locking holes 301, 306 of an intramedullary nail 300 inserted into the bone. A first bore hole may be drilled through the desired one of the first and second holes 301, 306 to accommodate the first bone screw 1′. As described above, the screwdriver 13′ is coupled to the first bone screw 1′ by receiving the head 2′ within the connector 132′. In an initial configuration, the longitudinal axis 130′ of the screwdriver 13′ is coaxially aligned with the first axis 6′ of the bone screw 1′. The first bone screw 1′ is screwed into the desired one of the holes 301, 306 and the first bore hole via the screwdriver 13′. Once the first bone screw 1′ has been inserted, as desired, the screwdriver 13′ is pivoted with respect to the first bone screw 1′ about the head 2′ until the channel 28′ thereof is coaxially aligned with the second axis 7′ of the through bore 9′. A second bore hole may be drilled into the bone through the channel 28′ and through bore 9′ to accommodate the second bone screw 50′. The second bone screw 50′ may then be guided through the channel 28′ and into through bore 9′ to be advanced into the second bore hole in the bone. It will be understood by those of skill in the art that the above-described steps may be repeated, as desired, until a desired number of first and second bone screws 1′, 50′ have been inserted into the bone.

Although the invention and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present invention.

It will be appreciated by those skilled in the art that various modifications and alterations of the invention can be made without departing from the broad scope of the appended claims. Some of these have been discussed above and others will be apparent to those skilled in the art.

What is claimed is:

1. A bone screw, comprising:
a screw shaft extending longitudinally along a screw axis;
a screw head extending from a proximal end of the screw shaft and configured to be releasably coupled to a surgical tool; and
a through hole defining a through hole axis and extending through the screw head, the through hole axis intersecting the screw axis at an acute angle α, the through hole being adapted to receive therein a second screw and tapering from a first end at a proximal end of the screw head to a second end opening to an outer surface of the screw head.

2. The bone screw according to claim 1, wherein the screw head includes a recess extending through a proximal end thereof, the recess configured to receive a portion of a surgical tool to be releasably coupled thereto and having a substantially circular edge centered at an intersection of the through hole axis and the screw axis.

3. The bone screw according to claim 2, further comprising a channel extending transversely through the screw head and defining a diametric channel axis, the channel being open at a proximal end of the bone screw.

4. The bone screw according to claim 1, wherein the through hole axis intersects the screw axis at a depth T>0 measured from the proximal end of the bone screw towards the screw shaft.

5. The bone screw according to claim 2, wherein the recess has a constriction at the proximal end of the screw head to facilitate a snap connection between the bone screw and the surgical instrument.

6. The bone screw according to claim 1, wherein the through hole comprises an internal thread.

7. The bone screw according to claim 6, wherein the internal thread has a thread pitch P and a threaded length L_{T} and wherein the ratio L_{T}/P is at least 2.0.

8. The bone screw according to claim 2, wherein the recess is substantially spherical.

9. The bone screw according to claim 2, wherein the recess includes a depression traversing the constriction and forming a wall portion forming a portion of one of a cylinder, cone and a prism an axis of which coincides with the through hole axis.

10. The bone screw according to claim 1, wherein the angle α is at least 10°.

11. The bone screw according to claim 1, wherein the angle α is at most 70°.

12. The bone screw according to claim 1, wherein in a cross-section orthogonal to the channel axis the transverse channel has a U-shape.

13. The bone screw according to claim 3, wherein the channel axis intersects the screw axis at the point at which the screw axis and the through hole axis intersect.

14. The bone screw according to claim 1, wherein the screw head of the bone screw includes an external thread configured such that the bone screw can be counter-sunk in a bone.

15. The bone screw according to claim 1, wherein the screw head of the bone screw has a longitudinal slot permitting the screw head to be radially elastically expanded.

16. The bone screw according to claim 1, wherein an exterior surface of the screw head is partially spherical and configured to be received within a portion of a surgical tool.

17. A system, comprising:
a first bone screw including:
a screw shaft extending longitudinally along a screw axis;
a screw head extending from a proximal end of the screw shaft and configured to be releasably coupled to a surgical tool; and
a through hole defining a through hole axis and extending through the screw head, the through hole axis intersecting the screw axis at an acute angle cc, the through hole being adapted to receive therein a second screw and tapering from a first end at a proximal end of the screw head to a second end opening to an outer surface of the screw head; and
a second bone screw sized and shaped to be inserted through the through hole of the first bone screw along the through hole axis.

18. The system according to claim 17, wherein the screw head includes a recess extending through a proximal end thereof, the recess configured to receive a portion of a surgical tool to be releasably coupled thereto and having a substantially circular edge centered at an intersection of the through hole axis and the screw axis.

19. The system according to claim 18, wherein the first bone screw further includes a channel extending transversely through the screw head and defining a diametric channel axis, the channel being open at a proximal end of the bone screw.

20. The system according to claim 17, wherein the second bone screw includes a threading along a head portion thereof for engaging a conical internal thread of the through hole.

21. The system according to claim 19, further comprising a screwdriver for a bone screw, the screw driver including a shaft extending longitudinally from a distal end to a proximal end, the distal end including a male connector configured to be coupled to the recess in the screw head of the first bone screw, the connector including a tip tapering towards the distal end of the screwdriver and two driving protrusions extending from opposite sides of the tip along a central axis extending orthogonally to the longitudinal axis of the screwdriver, wherein the driving protrusions engage the channel in the screw head of the first bone screw, and wherein the tip has a substantially circular periphery centered located on the longitudinal axis.

22. The system according to claim 21, wherein the screwdriver further comprises a longitudinal slot extending parallel to the longitudinal axis and which is open at the distal end permitting the connector to be radially elastically compressed, and wherein the tip has a constriction toward the shaft forming a curved contact shoulder.

23. The system according to claim 21, wherein the tip is formed as a portion of a sphere centered on the longitudinal axis.

24. The system according to claim 21, wherein the two driving protrusions are substantially cylindrical and wherein the central axis orthogonally cuts the longitudinal axis through the center of the tip.

25. The system according to claim 21, wherein the male connector further comprises an axial stop a distance T from the central axis towards the shaft so that the stop contacts the rear end of the first bone screw when the connector is coupled to the recess in the screw head.

26. The system according to claim 21, wherein the male connector further includes a nose projecting over the tip in a direction toward the distal end and at an acute angle with respect to the longitudinal axis of the screwdriver.

27. The system according to claim 21, further comprising a coaxial through bore extending through the shaft and the male connector and having an internal thread for engaging an external thread arranged on a locking pin insertable in the through bore so that the locking pin can be advanced towards the distal end of the screwdriver to prevent the tip from being radially compressed.

28. The system according to claim 18, further comprising an aiming guide connectable to the first bone screw, the aiming guide comprising a guide sleeve extending along a longitudinal axis, a coaxial through bore, a distal end including a male connector arranged at the distal end, the male connector configured to be coupled to the recess in the screw head of the first bone screw, wherein the connector includes a tip reducing in diameter towards the distal end of the aiming guide and two pins projecting from opposite sides of the tip along a central axis extending orthogonally to the longitudinal axis, wherein the tip has a substantially circular periphery centered on the longitudinal axis.

29. The system according to claim 28, wherein the aiming guide further comprises a longitudinal slot extending parallel to the longitudinal axis and which is open at the front end so that the connector is radially elastically compressible, and wherein the tip has a constriction towards the guide sleeve which forms a curved contact shoulder.

30. The system according to claim 28, wherein the tip is substantially spherical with a center on the longitudinal axis.

31. The system according to claim 28, wherein the two pins are substantially cylindrical and wherein the central axis orthogonally intersects the longitudinal axis at the center of the spherical tip.

32. The system according to claims 28, wherein between the tip and the guide sleeve a collar is arranged substantially coaxial with the longitudinal axis of the aiming guide, the collar being one of cylindrical and conical.

33. The system according to claim 32, wherein the collar has a radius smaller than a radius of the recess.

34. The system according to claims 17, further comprising a drill guide insertable into the through hole.

35. The system according to claim 34, wherein the drill guide has a conical tip.

36. The system according to claim 17, wherein the screw head has an exterior surface that is partially spherical.

37. The system according to claim 36, further comprising a screwdriver including a connector at a distal end thereof configured to receive the partially spherical screw head such that the first bone screw is pivotable between a first position in which the screw axis is substantially coaxial with a longitudinal axis of the screwdriver to a second position in which the through hole axis is coaxial with the longitudinal axis.

38. The system according to claim 37, wherein the screwdriver further includes a channel extending therethrough along the longitudinal axis thereof such that when the first bone screw is in the second position, the channel is configured to guide the second bone screw therethrough and into the through hole of the first bone screw.
